(19) United States
(12) Patent Application Publication
(10) Pub. No.: US 2025/0127580 A1
(43) Pub. Date: Apr. 24, 2025

(54) SURGICAL ROBOTIC SYSTEM AND METHOD FOR INSTRUMENT TRACKING AND VISUALIZATION IN NEAR INFRA-RED (NIR) IMAGING MODE USING NIR REFLECTION

(71) Applicant: Covidien LP, Mansfield, MA (US)

(72) Inventors: Matthew S. Pias, Cambridge, MA (US); Badr Elmaanaoui, Belmont, MA (US)

(21) Appl. No.: 18/889,536

(22) Filed: Sep. 19, 2024

Related U.S. Application Data
(60) Provisional application No. 63/592,244, filed on Oct. 23, 2023.

(51) Int. Cl. A61B 34/37; A61B 1/04; A61B 1/313; A61B 34/20; A61B 34/30; A61B 90/00

(52) U.S. Cl. CPC A61B 34/37; A61B 1/046; A61B 1/3132; A61B 2034/2048; A61B 2034/2057; A61B 2034/2065; A61B 2034/302; A61B 2090/364; A61B 2090/3941; A61B 2090/3979

(57) ABSTRACT

A surgical robotic system is paired with an imaging system that may operate in a plurality of imaging modes, including white light and near infrared (NIR) light. The robotic system includes a plurality of robotic arms, one of which controls a laparoscopic camera providing video data based on the selected imaging mode to an image processing device. One or more other arms control one or more corresponding robotic instruments, which include fiducial markers formed from a fluorescent material detectable in the NIR imaging mode. The markers allow for visualization of the instruments in the NIR imaging mode, which is monochromatic. The image processing device also generates masked or virtual overlays of the instrument and registers the overlays with the instruments in the monochromatic video feed, providing additional visualization. Registration of the overlays is performed using fiducial markers, which act like landmarks for aligning the overlay to the actual image of the instrument in the video feed.

### CROSS-REFERENCE TO RELATED APPLICATIONS

This application claims the benefit of, and priority to, U.S. Provisional Patent Application Ser. No. 63/592,244 filed on Oct. 23, 2023. The entire contents of the foregoing application are incorporated by reference herein.

### BACKGROUND

Surgical robotic systems are currently being used in a variety of surgical procedures, including minimally invasive surgical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. A laparoscopic camera, which is also held by one of the robotic arms, is inserted into the patient to image the surgical site.

The laparoscopic camera may operate in a variety of imaging modes including conventional color, or white light, mode and fluorescence mode. In conventional white light mode, light in the visible spectral range is used to illuminate the tissue surface under observation. Light reflected by the tissue passes through a suitable lens system and is incident on an image sensor built into or attached to the endoscope. The electrical signals from the image sensor are processed into a full color video image which can be displayed on a video monitor or stored in a memory.

In fluorescence mode, fluorescence excitation light excites fluorophores in the tissue, which emit fluorescence light at an emission wavelength, which is typically greater than the excitation wavelength. Fluorescence light from the tissue passes through a suitable lens system and is incident on the image sensor. The electrical signals from the image sensor are processed into a fluorescence video image which can be displayed on a video monitor, either separately or combined with the color video image.

The fluorescence excitation and emission wavelengths depend upon the type of fluorophores being excited. In the case of exogenously applied fluorophores, such as a fluorescent dye (e.g., indocyanine green (ICG)) the band of excitation wavelengths may be located anywhere in the range from the ultraviolet (UV) to the near infra-red (NIR) and the emission wavelength band anywhere from the visible to the NIR. For fluorophores endogenous to tissue, the band of excitation and emission wavelengths are more limited (excitation from the UV to the green part of the visible spectrum, emission from the blue/green light to the NIR). Fluorescence imaging may be used to identify blood vessels, cancer cells, and other tissue types. White light and fluorescence imaging modes may be combined in a variety of ways. Camera manufacturers offer various imaging modes to provide surgeons additional insight into the structures and tools used during laparoscopic or surgical procedures. Certain modes, which enhance NIR light, result in low visibility of objects, e.g., instruments, that do not fluoresce. Since the instruments are not sufficiently visible in monochromatic imaging mode, the users are forced to switch imaging modes to properly locate instruments in relation to fluorescing tissue, especially if instruments need to be repositioned.

In monochromatic mode the surgical instruments are not visible within the endoscopic field of view (FOV) due to low fluorescence of instrument material. However, due to the high contrast of monochromatic mode favoring clear visualization of structures perfusing ICG, surgeons prefer this view to visualize anatomy. Therefore, instrument movement is disallowed as a safety precaution while displaying in monochromatic mode as instruments cannot be seen within the endoscopic FOV as they do not fluoresce under NIR light. Therefore, surgeons do not have a clear understanding of where the instruments are in relation to the fluorescent structures shown in monochrome mode and will not be able to move them until returning to a mode with white light enabled.

### SUMMARY

The present disclosure provides a surgical robotic system that includes an imaging system that is operable in a plurality of imaging modes. The robotic system may include one or more robotic arms each holding an instrument or a laparoscopic camera of the imaging system. The imaging system is configured to obtain white color and NIR images of the tissue using fluorophores from a fluorescent dye, e.g., ICG. The imaging system is configured to combine the white and NIR images in an overlay mode, during which the regular white light image is combined with the NIR/ICG data to generate an overlay image. In overlay mode, the imaging system may be configured to display the NIR image using visible light depending on user preferences and application, e.g., the NIR/ICG data can be displayed as a green or blue overlay. In intensity map mode, the imaging system displays the intensity of the NIR/ICG signal using a color scale in an overlay image. In a monochromatic mode, the NIR/ICG signal alone is displayed in white on a black background to achieve the greatest possible differentiation.

The imaging system is programmed to operate with instruments that include fiducial markers to enhance their visualization. Any suitable fluorescence material may be used and may be applied as a coating on a portion or entirety of the instrument. The material may also be applied as discrete fiducial markers highlighting points of the instrument's structure. In embodiments, the material may be applied on attachments (e.g., stickers, clips, or fasteners), which may be removable to prevent damage during instrument reprocessing, mechanical interference when not in use, etc. formulated such that enough fluorescence occurs to enable visualization but not so much fluorescence such that it washes out the fluorescing anatomy.

According to one embodiment of the present disclosure, a surgical robotic system is disclosed. The surgical robotic system includes a robotic arm having an instrument drive unit. The system also includes a surgical instrument having a plurality of fluorescent fiducial markers. The surgical instrument is coupled to and actuatable by the instrument drive unit. The system further includes a laparoscopic camera for capturing a video feed of the surgical instrument. The system additionally includes an image processing device coupled to the laparoscopic camera. The image processing device is operatable in a white light imaging mode and a low visibility imaging mode. The system additionally includes a controller for processing the video feed of the surgical instrument in the low visibility imaging mode to detect the plurality of fluorescent fiducial markers. The controller further generates an overlay of the surgical instrument and renders the overlay, while in the low visibility imaging mode, on a portion of the video feed having the surgical instrument, based on locations of the identified plurality of fluorescent fiducial markers. The system also includes a screen for displaying the video feed in the low visibility imaging mode with the overlay.

Implementations of the above embodiment may include one or more of the following features. According to one aspect of the above embodiment, the laparoscopic camera captures white light and near infrared (NIR) light images. The low visibility imaging mode may be a monochromatic NIR mode. The overlay may be a virtual overlay and may be one of a line model, a mesh model, or a 3D surface model of the instrument. The overlay may be a masked overlay of the instrument. The controller may further generate the masked overlay of the instrument from the video feed while in the white light imaging mode. The system may also include a surgeon console including a handle controller for receiving user input, where the instrument may be actuated by the instrument drive unit in response to the user input. The controller may further track the position of the surgical instrument and update a location of the overlay on the video feed based on the position of the surgical instrument. The controller may further track movement of the surgical instrument based on kinematics data of the robotic arm and the plurality of plurality of fluorescent fiducial markers.

According to another embodiment of the present disclosure, a surgical robotic system is disclosed. The surgical robotic system includes a robotic arm having an instrument drive unit. The system also includes a surgical instrument having a plurality of fluorescent fiducial markers. The surgical instrument is coupled to and actuatable by the instrument drive unit. The system additionally includes a laparoscopic camera for capturing a video feed of the surgical instrument. The system also includes an image processing device coupled to the laparoscopic camera. The image processing device is operatable in a white light imaging mode and a low visibility imaging mode. The system also includes a controller for processing the video feed of the surgical instrument in the low visibility imaging mode to detect the plurality of fluorescent fiducial markers. The controller also selects an overlay for the instrument from a plurality of overlays. The controller further generates the selected overlay of the surgical instrument and renders the selected overlay, while in the low visibility imaging mode, on a portion of the video feed may include the surgical instrument based on locations of the identified plurality of fluorescent fiducial markers. The system also includes a screen for displaying the video feed in the low visibility imaging mode with the overlay.

Implementations of the above embodiment may include one or more of the following features. According to one aspect of the above embodiment, the plurality of overlays may include a virtual overlay and a masked overlay. The virtual overlay may be one of a line model, a mesh model, or a 3D surface model of the instrument. The controller may further generate the masked overlay of the instrument from the video feed while in the white light imaging mode. The system may also include a surgeon console including a handle controller for receiving user input, where the instrument may be actuated by the instrument drive unit in response to the user input. The controller may further track a position of the surgical instrument and update a location of the overlay on the video feed based on the position of the surgical instrument. The controller may also track movement of the surgical instrument based on kinematics data of the robotic arm and the plurality of plurality of fluorescent fiducial markers.

According to a further embodiment of the present disclosure, a method for controlling a surgical robotic system is disclosed. The method also includes capturing a video feed of a surgical instrument through a laparoscopic camera, where the instrument includes a plurality of fluorescent fiducial markers. The method also includes operating an image processing device coupled to the laparoscopic camera in a white light imaging mode and a low visibility imaging mode. The method may include processing the video feed of the surgical instrument in the low visibility imaging mode to detect the plurality of fluorescent fiducial markers. The method further includes generating an overlay of the surgical instrument. The method additionally includes rendering the overlay while in the low visibility imaging mode, on a portion of the video feed showing the surgical instrument, based on locations of the identified plurality of fluorescent fiducial markers. The method also includes displaying on a screen the video feed in the low visibility imaging mode with the overlay.

Implementations of the above embodiment may include one or more of the following features. According to one aspect of the above embodiment, the method may also include: receiving user input at a surgeon console having a handle controller, where the instrument is coupled to a robotic arm through an instrument drive unit; and actuating the instrument through the instrument drive unit in response to the user input. The method may also include: tracking a position of the surgical instrument; and updating a location of the overlay on the video feed based on the position of the surgical instrument.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a schematic diagram of a system for determining phases of a surgical procedure according to an embodiment of the present disclosure;

FIG. 7 is a perspective view of an imaging system according to an embodiment of the present disclosure;

FIG. 8 is a screenshot of a graphical user interface (GUI) for selecting an imaging mode according to an embodiment of the present disclosure;

FIGS. 9A and B show a flow chart of a method for instrument tracking and visualization in near infra-red imaging mode according to an embodiment of the present disclosure;

FIG. 10 is a screenshot of a surgeon screen displaying a video captured by a laparoscopic camera of a surgical instrument having NIR reflective fiducial markers in a color (i.e., white light) imaging mode according to an embodiment of the present disclosure;

FIG. 11 is a screenshot of the surgeon screen displaying the video of the surgical instrument having NIR reflective fiducial markers in a monochromatic NIR imaging mode according to an embodiment of the present disclosure;

FIG. 12 is a screenshot of the surgeon screen displaying the video of the surgical instrument having NIR reflective fiducial markers in the monochromatic NIR imaging mode and in color imaging mode showing correspondence of the fiducial markers in each mode; and

FIG. 13 is a screenshot of the surgeon screen displaying the video of the surgical instrument having alternative NIR reflective fiducial markers in a monochromatic NIR imaging mode according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

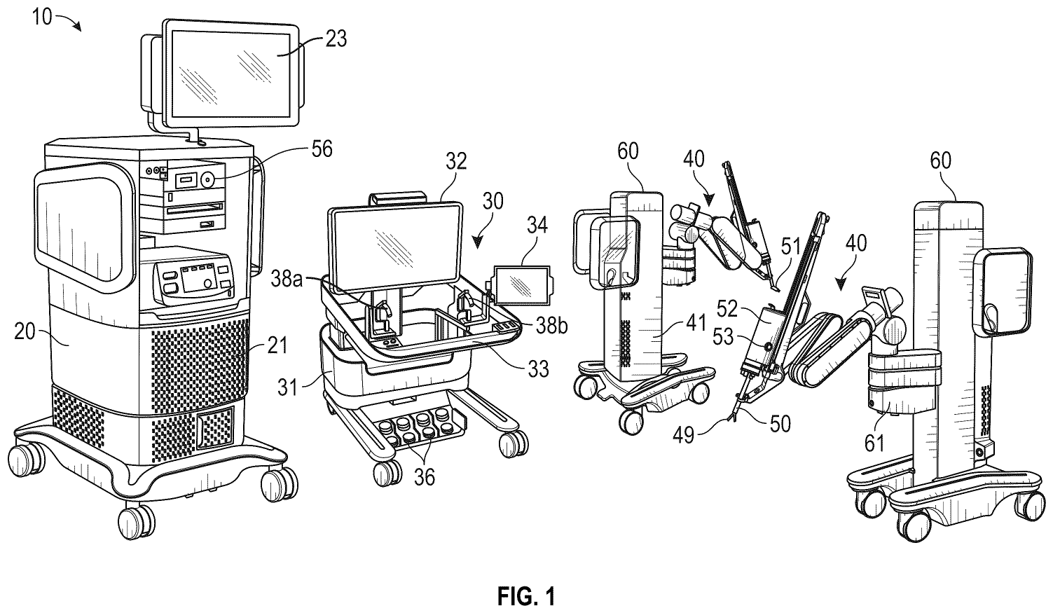

With reference to FIG. 1, a surgical robotic system 10 includes a control tower 20, which is connected to all the components of the surgical robotic system 10 including a surgeon console 30 and one or more mobile carts 60. Each of the mobile carts 60 includes a robotic arm 40 having a surgical instrument 50 removably coupled thereto. The robotic arms 40 also couple to the mobile carts 60. The robotic system 10 may include any number of mobile carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical or ultrasonic instrument, such as a forceps configured to seal tissue by compressing tissue between jaw members and applying electrosurgical current or ultrasonic vibrations via an ultrasonic transducer to the tissue. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue. The system also includes an electrosurgical generator configured to output electrosurgical (e.g., monopolar or bipolar) or ultrasonic energy in a variety of operating modes, such as coagulation, cutting, sealing, etc. Suitable generators include a Valleylab™ FT10 Energy Platform available from Medtronic of Minneapolis, MN.

One of the robotic arms 40 may include a laparoscopic camera 51 configured to capture video of the surgical site. The laparoscopic camera 51 may be a stereoscopic camera configured to capture two side-by-side (i.e., left and right) images of the surgical site to produce a video stream of the surgical scene. The laparoscopic camera 51 is coupled to an image processing device 56, which may be disposed within the control tower 20. The image processing device 56 may be any computing device configured to receive the video feed from the laparoscopic camera 51 and output the processed video stream.

The surgeon console 30 includes a first, i.e., surgeon, screen 32, which displays a video feed of the surgical site provided by camera 51 of the surgical instrument 50 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

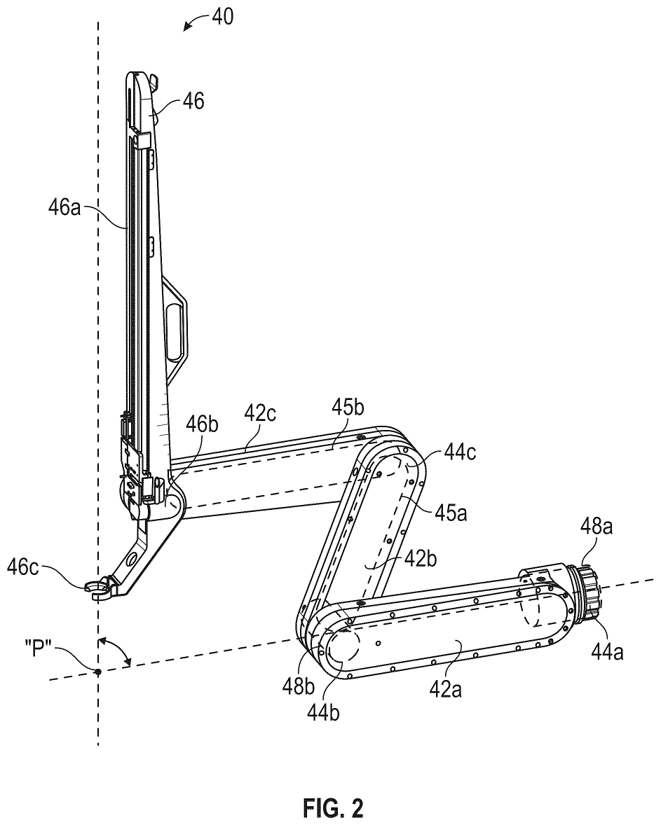

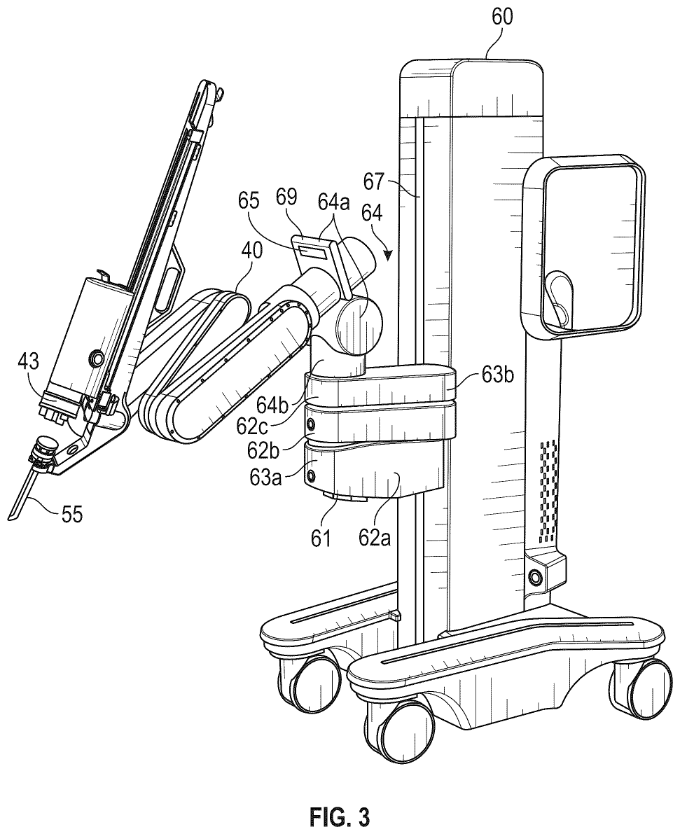

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting the robotic arm 40. The lift 67 allows for vertical movement of the setup arm 61. The mobile cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During laparoscopic procedures, the instrument 50 may be inserted through a laparoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

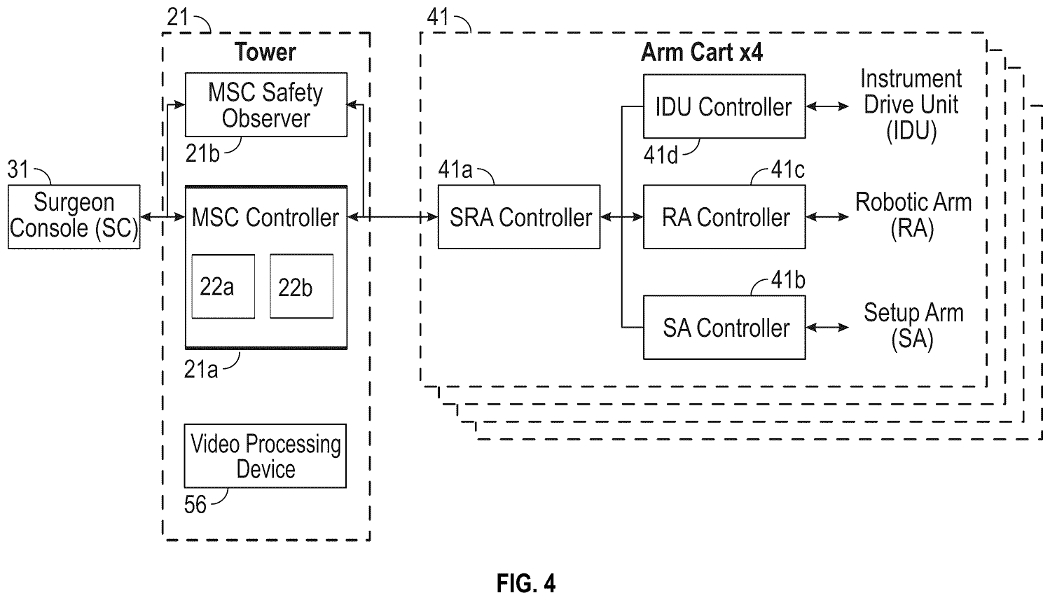

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

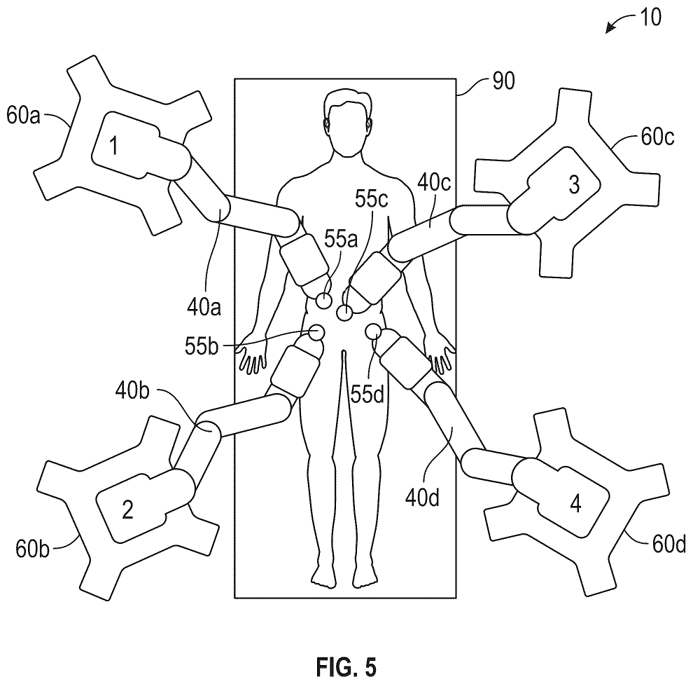

With reference to FIG. 5, the surgical robotic system 10 is set up around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

A surgical procedure may include multiple phases, and each phase may include one or more surgical actions. As used herein, the term “phase” represents a surgical event that is composed of a series of steps (e.g., closure). A “surgical action” may include an incision, a compression, a stapling, a clipping, a suturing, a cauterization, a sealing, or any other such actions performed to complete a phase in the surgical procedure. A “step” refers to the completion of a named surgical objective (e.g., hemostasis). During each step, certain surgical instruments 50 (e.g., forceps) are used to achieve a specific objective by performing one or more surgical actions.

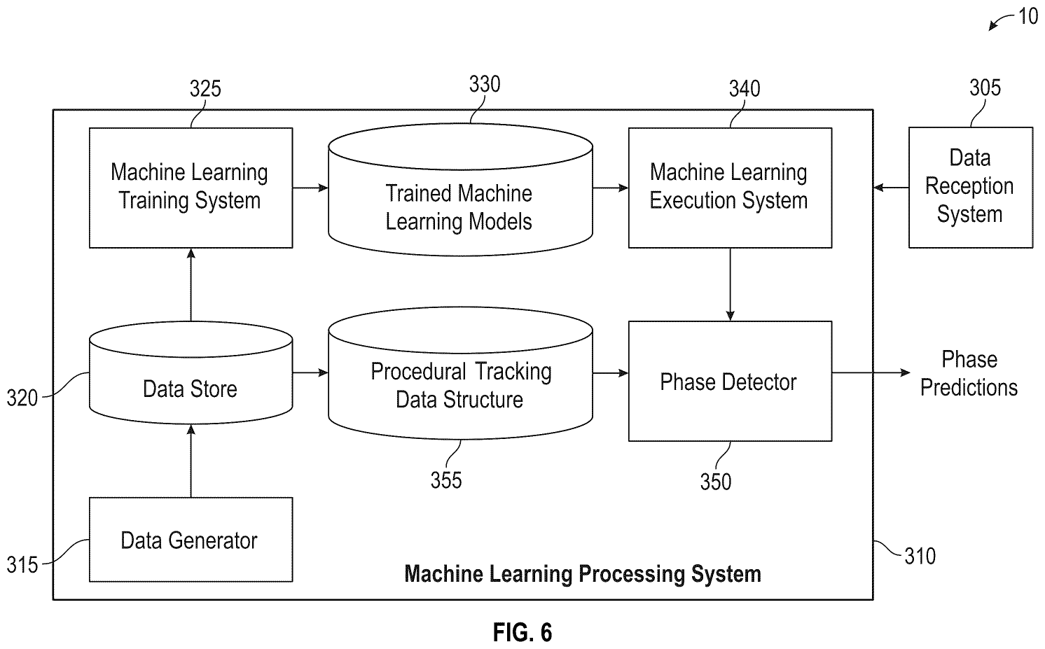

With reference to FIG. 6, the surgical robotic system 10 may include a machine learning (ML) processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual or masked images, or record the video data from the image processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure. Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of surgical procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the surgical procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine when to switch between various imaging modes as described below.

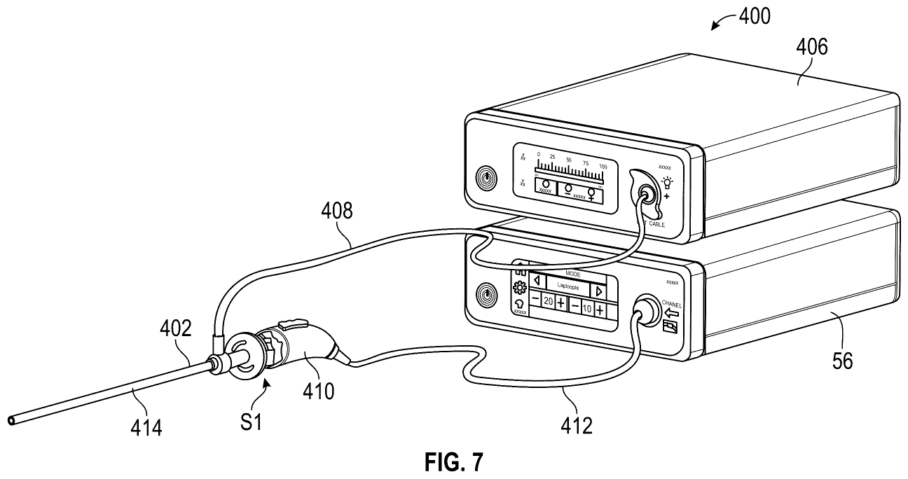

With reference to FIG. 7, the surgical robotic system 10 also includes an imaging system 400, in which the laparoscopic camera 51 coupled to the image processing device 56. The laparoscopic camera 51 includes a laparoscope 402 having a longitudinal shaft 414 with a plurality of optical components (not shown), such as lenses, mirrors, prisms, and the like disposed in the longitudinal shaft 414. The laparoscope 402 is coupled to a combined light source 406 via an optical cable 408. The light source 406 may include a white light source (not shown) and an NIR light source (not shown), which may be light emitting diodes or any other suitable light sources. The NIR light source may be a laser or any other suitable light source. The optical cable 408 may include one or more optical fibers for transmitting the white and NIR light, which illuminates the tissue under observation by the laparoscope 402. The laparoscope 402 collects the reflected white and NIR light and transmits the same to a camera assembly 410, which is coupled to a proximal end portion of the laparoscope 402. The laparoscope 402 may be any conventional laparoscopic configured to transmit and collect white and NIR light.

The camera assembly 410 is configured to separate fluorescence wavelength from undesired components of the light spectrum to specific sensors. In particular, the camera assembly includes a white (e.g., visible) light (VIS) sensor and an IR sensor and is configured to separate and transmit white light to the VIS sensor and fluorescence IR light to the IR sensor. The VIS sensor and the IR sensor may be a complementary metal oxide semiconductor (CMOS) image sensors having any desired resolution, which in embodiments may be 4K, UHD, etc.

The camera assembly 410 is coupled to the image processing device 56 via a transmission cable 412. The image processing device 56 is configured to receive the image data signals, process the raw image data from the camera assembly 410, and generate blended white light and NIR images for recording and/or real-time display. The image processing device 56 also processes the image data signals and outputs the same to any of the display screens 23, 32, 34 of the surgical robotic system 10, through any suitable a video output port, such as a DISPLAYPORT™, HDMI®, etc., that is capable of transmitting processed images at any desired resolution, display rates, and/or bandwidth.

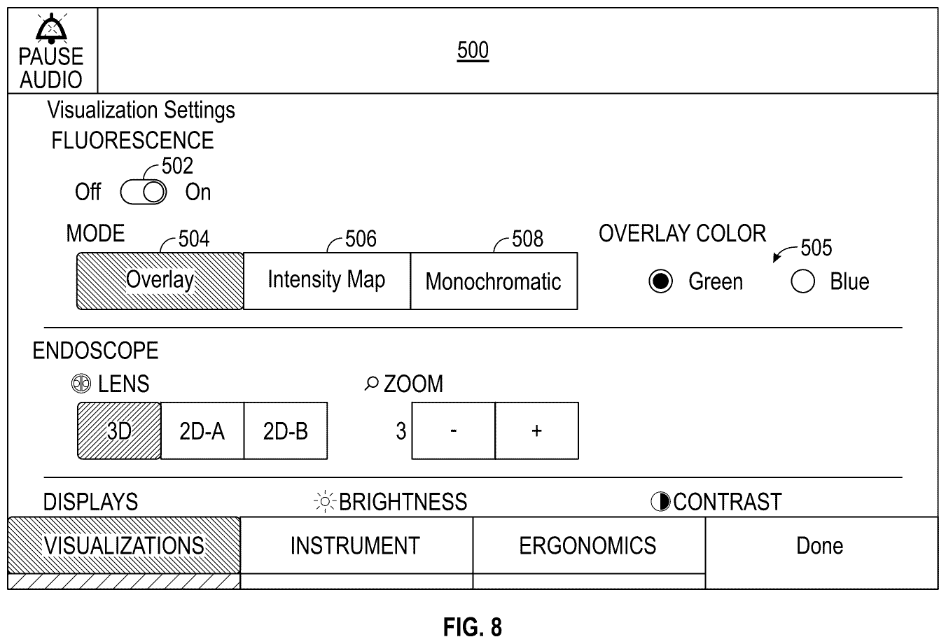

FIG. 8 shows a GUI 500 for controlling the imaging system 400, which may be displayed on the display screens 23, 32, 34 of the surgical robotic system 10. The GUI 500 includes options for controlling fluorescence settings, including turning fluorescence (e.g., NIR detection) on or off via toggle 502. Once fluorescence is selected, the user may also select from a plurality of imaging modes that visualize NIR light. An overlay mode is selectable via a button 504. In overlay mode, the imaging system 400 combines the white and NIR images, during which the regular white light image is combined with the NIR/ICG data to generate an overlay image. In this mode, the imaging system may be configured to display the NIR light in a visible light depending on user preferences and application, the NIR/ICG data can be displayed as a green or blue overlay, which is selectable via a menu 505. In intensity map mode, selectable via button 506, the imaging system displays the intensity of the NIR/ICG signal using a color scale in an overlay image. In monochromatic mode, selectable via button 508, the NIR/ICG signal alone is displayed in white on a black background to achieve the greatest possible differentiation as shown in FIG. 11. In embodiments, the modes may be also selectable via one or more foot pedals 36 associated with mode selection, e.g., one foot pedal cycles through each of the NIR modes.

The imaging system 400 is used to detect and track instrument 50 having a plurality of fiducial markers 700 that are formed from a fluorescent material. Suitable fluorescent materials include a composition having a polymeric matrix and a fluorophore dye, such as rhodamine, indocyanine green or dansyl chloride. The polymeric matrix may include one or more hydrophobic, biocompatible polymers including, but not limited to, poly(methyl methacrylate), poly(ethyl methacrylate), poly(propyl methacrylate), poly(butyl methacrylate), poly(methyl methacrylate-co-methacrylic acid), poly(lactide-co-glycolide), polylactic acid, polyglycolic acid, polycaprolacton, cellulose triacetate, nitrocellulose, polydimethylsiloxane, poly(ethylene terephthalate), polycarbonate, polyethylene, ethylene vinyl acetate copolymer, polyurethane, polystyrene, and copolymers thereof with poly(ethylene glycol).

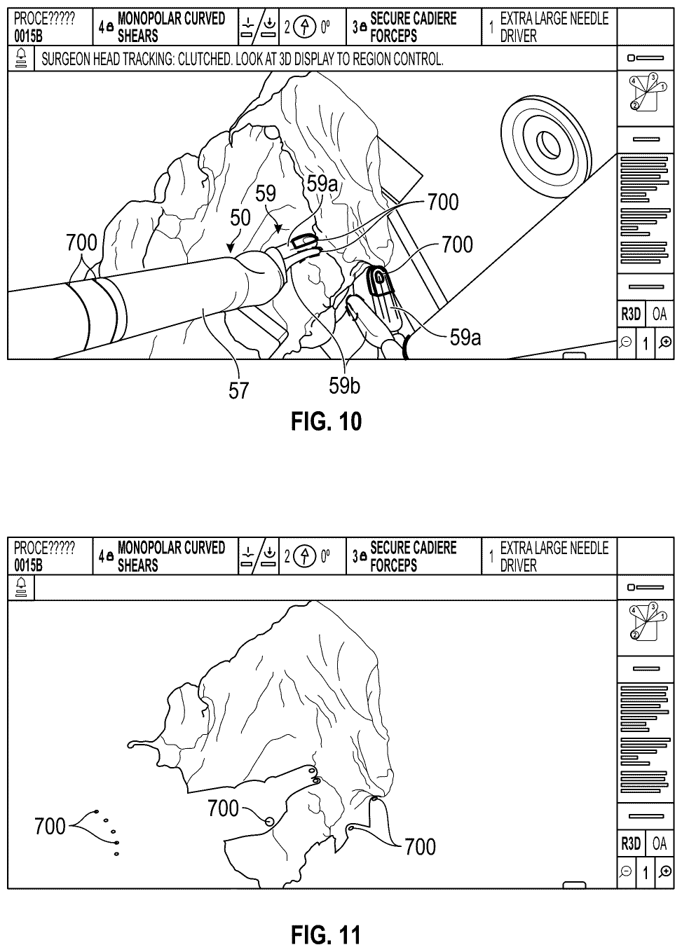

The fiducial markers 700 may be applied to any portion of the instrument 50, such as a shaft 57 or end effector 59 having a pair of jaws 59a and 59b. The fiducial markers 700 may have any suitable shape, such as dots as shown in FIG. 11 or more complex designs outlining contours of the instrument 50, e.g., the jaws 59a and 59b, shaft 57, etc. as shown in FIG. 13. The fiducial markers 700 may be enhanced (e.g., include a color marker) to be visible under white light illumination as shown in FIG. 10. Fluorescent fiducial markers 700 increase contrast of the instruments 50 in the monochromatic imaging mode thereby making the instrument 50 easily identifiable to the naked eye as well as when using computer vision algorithms. In particular, the higher contrast of the images of fluorescent materials lowers the processing requirements of the images when identifying the fiducial markers 700 when compared to image analysis of color (e.g., white light) images.

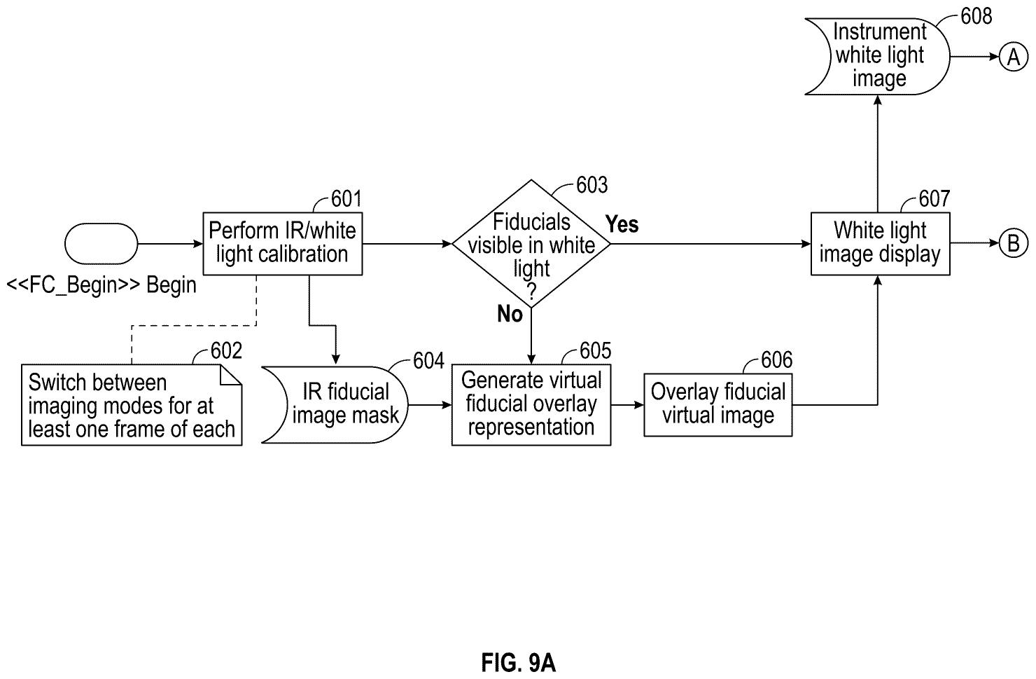

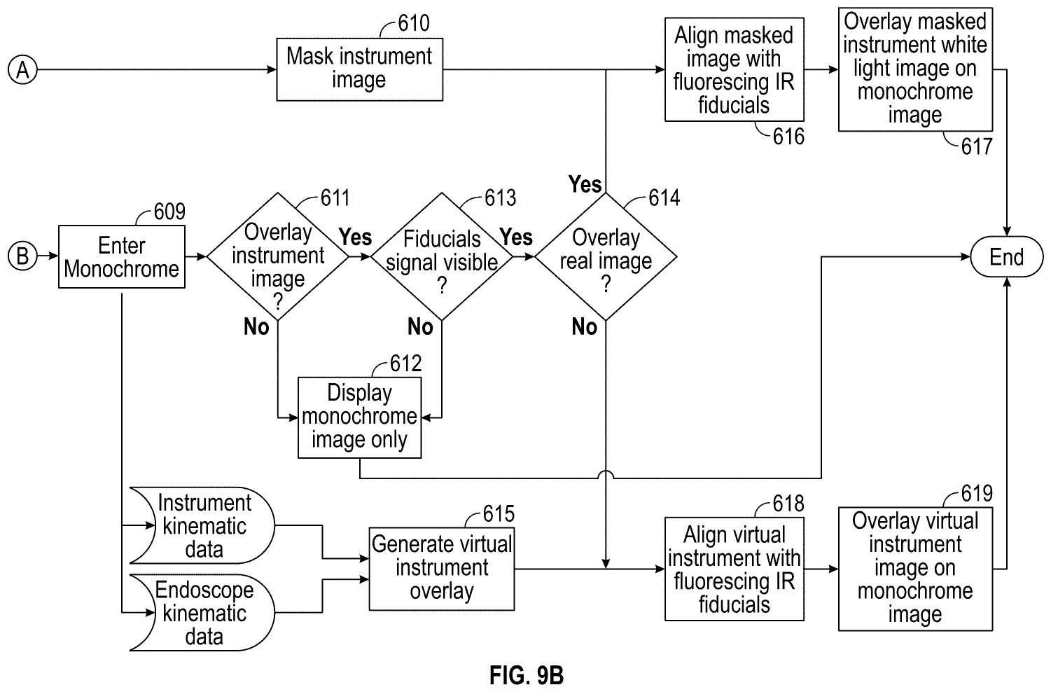

FIGS. 9A and 9B show a method of operating the surgical robotic system 10 along with the imaging system 400, to enable instrument tracking and visualization in NIR imaging mode. The method may be embodied as software instructions executable by any controller of system 10, e.g., main controller 21a. At step 601, the laparoscopic camera 51 is calibrated or calibration data is received if calibration was performed previously. The camera 51 may be a stereoscopic camera that may be calibrated prior to or during use in a surgical setting. Calibration may be performed using a calibration pattern, which may be a checkerboard pattern of black and white or fluorescent shapes, e.g., squares. The calibration may include obtaining a plurality of images at different poses and orientations and providing the images as input to the image processing device 56, which outputs calibration parameters for use by the camera user. Calibration parameters may include one or more intrinsic and/or extrinsic parameters such as position of the principal point, focal length, skew, sensor scale, distortion coefficients, rotation matrix, translation vector, and the like. The calibration parameters may be stored in a memory of the camera 51 and loaded by the image processing device 56 upon connecting to the camera 51.

At step 602, calibration may be performed prior to use of the imaging system 400 and the process includes switching the image processing device 56 between a white light mode and NIR imaging mode to calibrate the camera 51 in each of the corresponding modes.

At step 603, the image processing device 56 is operated in the white light mode and determines whether the fiducial markers 700 are visible in the white light imaging mode. In addition to including fluorescent material, the fiducial markers 700 may include visual enhancements (e.g., color patterns) that are visible under white light illumination as shown in FIG. 10. The image processing device 56 may make the determination using an ML/AI computer vision algorithm, which runs in the background and detects the fiducial markers from the white light or NIR imaging data. The ML/AI algorithm may be based on statistical ML that is configured to a develop a statistical model and draw inferences therefrom. As more training data is provided, the system adjusts the statistical model and improves its ability to analyze or make predictions. Suitable statistical ML models include, but are not limited to, linear regression, logistic regression, decision trees, random forest, Naïve Bayes, ensemble methods, support vector machines, K-Nearest Neighbor, and the like. In further embodiments, the ML/AI algorithm may be a deep learning algorithm that incorporates neural networks in successive layers. Suitable deep learning models include, but are not limited to, convolutional neural network, recurrent neural network, deep reinforcement network, deep belief network, transformer network, and the like. The input provided to train the models may be previously collected data, including images of instruments fiducial markers 700. If the fiducial markers 700 are visible in the white light mode, as determined by the image processing device 56, then the image processing device 56 outputs the white light video feed in the white light mode. If not, then the method proceeds to step 605 to generate masked fiducial marker overlay.

In parallel with step 603, at step 604, the image processing device 56 also executes NIR fiducial image masking mode, which is used to generate masked images of the fiducial markers 700 in white light mode. Since the fiducial markers 700 are only visible in NIR mode, the NIR fiducial image masking mode visualizes the fiducial markers 700 in the white light mode by outputting an overlay of the markers 700. Initially, at step 604 the image processing device 56 identifies the fiducial markers 700 in the NIR video feed captured by the camera 51 and generates a masked fiducial marker overlay.

At step 605, the image processing device 56 generates masked fiducial marker overlays by using image masking. The image processing device 56 extracts the images of the fiducial markers 700 from the NIR images and overlays the same on the white light video feed at step 606. At step 607, the white light video feed is displayed on the screen 32 including the fiducial markers 700 either directly, if the markers are visible as described above and shown in FIG. 10, or as overlays, if the markers are not visible under white light.

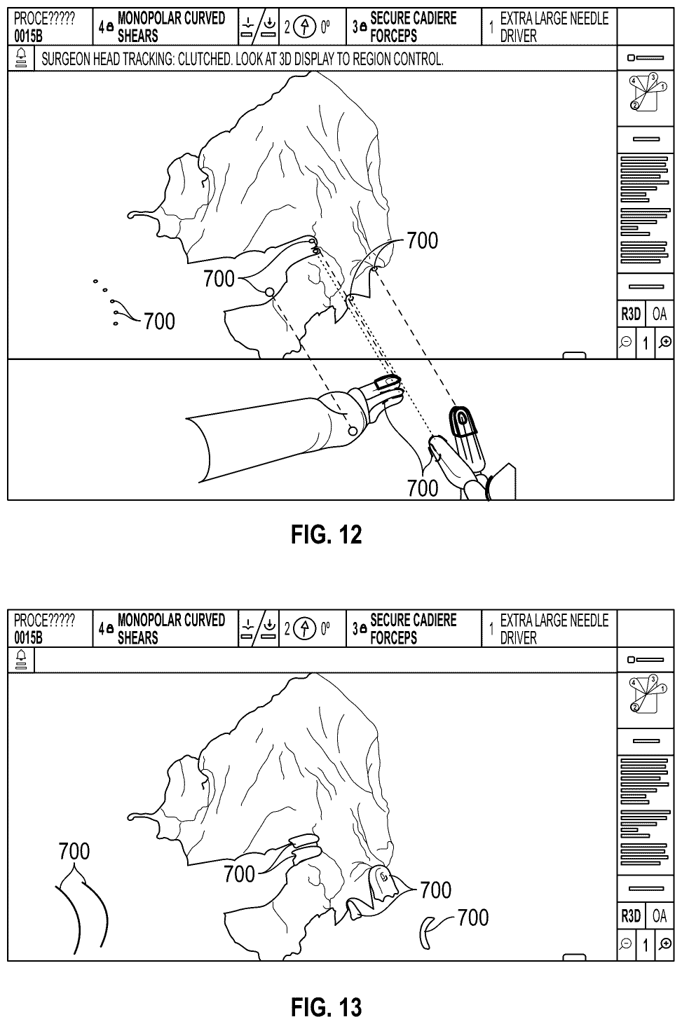

At step 609, the image processing device 56 is switched to the NIR monochromatic imaging mode. In parallel with step 609, at step 608, the image processing device 56 executes white light instrument masking mode, which is used to generate masked images of the instrument 50 in the NIR monochromatic imaging mode. Since most of the instrument 50, aside from fluorescent materials of the markers 700, is only visible under white light, the instrument masking mode may be used to visualize the instrument 50 in the NIR monochromatic imaging mode by outputting an overlay of the instrument 50 as shown in FIG. 12. At step 610 the image processing device 56 identifies the instrument 50 in the white light video feed captured by the camera 51 and generates a masked instrument overlay. A computer vision AI/ML algorithm may be used to identify the instrument 50 in the images. The image processing device 56 generates masked instrument overlay by using image masking.

At step 611, the image processing device 56 determines whether the virtual image of the instrument 50 is to be overlayed in the monochromatic imaging mode, i.e., over the monochromatic video feed. The determination may be based on the detected phase of the surgical procedure (e.g., output of phase detector 350) or in response to a user request. The user input may be provided in response to a prompt output by the image processing device 56, which occurs upon switching to the NIR monochromatic video feed. If the determination is that the overlay image of the instrument 50 is not to be displayed, then at step 612, the image processing device 56 outputs only the monochromatic image as shown in FIGS. 11-13.

If the determination is that the overlay image of the instrument 50 is to be displayed, then at step 613 the image processing device 56 determines whether the fiducial markers 700 are visible in the NIR image as shown in FIGS. 11-13. If the fiducial markers 700 are not visible or detectable by the image processing device 56 using a computer vision algorithm, then at step 612, the image processing device 56 outputs only the monochromatic image as shown in FIGS. 11-13. However, if the fiducial markers 700 are visible, then at step 614 the image processing device 56 proceeds to overlay the overlay (e.g., either masked or virtual) image of the instrument 50.

Upon entering the monochromatic imaging mode, the image processing device 56 also generates a virtual instrument overlay at step 615. The overlay of the instrument 50 may be a 3D generated model, (e.g., line model, mesh model, 3D surface model, etc.) based on white light images and/or 3D CAD model data. The white light overlay of the instrument 50 allows for movement of the camera 51 where the overlay is masked from the white light image. A computer generated overlay may be used where both the camera 51 and instrument 50 are moved, and as such allows for greater accuracy of the overlay in this scenario without leaving monochrome mode.

The image processing device receives kinematic data for each robotic arm 40 that is moving and controlling the instrument 50 and the camera 51. The kinematic data is used to move the overlay on the video feed along with the instrument 50 and/or the camera 51 as described below such that the overlay follows, i.e., tracks the movement of the instrument 50 and/or the camera 51 regardless of whether they are visible (i.e., in NIR imaging mode).

Returning to step 614, the image processing device 56 determines whether to display the masked (i.e., real) image of the instrument 50. The determination may be based on the detected phase of the surgical procedure (e.g., output of phase detector 350) or in response to a user request. The user input may be provided in response to a prompt output by the image processing device 56.

If the determination at step 614 is that the overlay image of the instrument 50 to be displayed is the masked (i.e., real) image, then at step 616 the image processing device 56 aligns the masked instrument overlay over the image of the instrument 50 in the monochromatic video feed. Alignment may include registration of the overlay with the instrument 50. The fiducials markers 700 may be used along with other surface features of the instrument 50 as tether points for registering and then rendering the overlay as shown in FIG. 12.

Once the overlay is registered, the overlay is continuously updated, i.e., adjusted and refreshed as the instrument 50 is moved within the view of the camera 51. Overlay update is performed using the ML/AI computer vision algorithm and kinematic data from joint and position sensors of the motors of the robotic arm 40 and/or the IDU 52. The kinematic data, which includes movement vectors and distances, is used to transform and update the overlay as well as move the overlay while the instrument 50 is moved. Thus, the original overlay generated is transformed and is regenerated based on the movement of the instrument 50 and/or the camera 51 since the overlay was previously mapped to a known coordinate system. The kinematic data may be used in combination with the ML/AI computer vision algorithm, which detects changes in position of the instruments 50 as captured by the camera 51. The overlay update process is performed continuously while the instrument 50 is moved until the overlay process is disabled, e.g., by exiting the low visibility, monochromatic mode. At step 617, the overlay is displayed on the monochromatic image covering the instrument 50 as shown in FIG. 12.

If the determination at step 614 is that the overlay image of the instrument 50 to be displayed is the virtual image generated at step 615, then at step 618 the image processing device 56 aligns the virtual instrument overlay over the image of the instrument 50 in the monochromatic video feed. Alignment may include registration of the overlay with the instrument 50. The fiducials markers 700 may be used along with other surface features of the instrument 50 as tether points for registering and then rendering the overlay in the same manner as shown in FIG. 12 for the masked instrument overlay. Once the overlay is registered the overlay is continuously updated in the same manner described at step 616. At step 619, the overlay is displayed on the monochromatic image covering the instrument 50 as shown in FIG. 12.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including an instrument drive unit;
a surgical instrument including a plurality of fluorescent fiducial markers, wherein the surgical instrument is coupled to and actuatable by the instrument drive unit;
a laparoscopic camera for capturing a video feed of the surgical instrument;
an image processing device coupled to the laparoscopic camera, the image processing device operatable in a white light imaging mode and a low visibility imaging mode;
a controller for:
processing the video feed of the surgical instrument in the low visibility imaging mode to detect the plurality of fluorescent fiducial markers;
generating an overlay of the surgical instrument; and
rendering the overlay, while in the low visibility imaging mode, on a portion of the video feed including the surgical instrument, based on locations of the identified plurality of fluorescent fiducial markers; and
a screen for displaying the video feed in the low visibility imaging mode with the overlay.

2. The surgical robotic system according to claim 1, wherein the laparoscopic camera captures white light and near infrared (NIR) light images.

3. The surgical robotic system according to claim 2, wherein the low visibility imaging mode is a monochromatic NIR mode.

4. The surgical robotic system according to claim 1, wherein the overlay is a virtual overlay and includes at least one of a line model, a mesh model, or a 3D surface model of the instrument.

5. The surgical robotic system according to claim 1, wherein the overlay is a masked overlay of the instrument.

6. The surgical robotic system according to claim 5, wherein the controller further generates the masked overlay of the instrument from the video feed while in the white light imaging mode.

7. The surgical robotic system according to claim 1, further comprising:
a surgeon console including a handle controller for receiving user input, wherein the instrument is actuated by the instrument drive unit in response to the user input.

8. The surgical robotic system according to claim 7, wherein the controller further tracks a position of the surgical instrument and updates a location of the overlay on the video feed based on the position of the surgical instrument.

9. The surgical robotic system according to claim 8, wherein the controller tracks movement of the surgical instrument based on kinematics data of the robotic arm and the plurality of plurality of fluorescent fiducial markers.

10. A surgical robotic system comprising:
a robotic arm including an instrument drive unit;
a surgical instrument including a plurality of fluorescent fiducial markers, wherein the surgical instrument is coupled to and actuatable by the instrument drive unit;
a laparoscopic camera for capturing a video feed of the surgical instrument;
an image processing device coupled to the laparoscopic camera, the image processing device operatable in a white light imaging mode and a low visibility imaging mode;
a controller for:
processing the video feed of the surgical instrument in the low visibility imaging mode to detect the plurality of fluorescent fiducial markers;
selecting an overlay for the instrument from a plurality of overlays;
generating the selected overlay of the surgical instrument; and
rendering the selected overlay, while in the low visibility imaging mode, on a portion of the video feed including the surgical instrument based on locations of the identified plurality of fluorescent fiducial markers; and
a screen for displaying the video feed in the low visibility imaging mode with the overlay.

11. The surgical robotic system according to claim 10, wherein the plurality of overlays includes a virtual overlay and a masked overlay.

12. The surgical robotic system according to claim 11, wherein the virtual overlay is at least one of a line model, a mesh model, or a 3D surface model of the instrument.

13. The surgical robotic system according to claim 11, wherein the controller further generates the masked overlay of the instrument from the video feed while in the white light imaging mode.

14. The surgical robotic system according to claim 10, further comprising:
a surgeon console including a handle controller for receiving user input, wherein the instrument is actuated by the instrument drive unit in response to the user input.

15. The surgical robotic system according to claim 14, wherein the controller further tracks a position of the surgical instrument and updates a location of the overlay on the video feed based on the position of the surgical instrument.

16. The surgical robotic system according to claim 15, wherein the controller tracks movement of the surgical instrument based on kinematics data of the robotic arm and the plurality of plurality of fluorescent fiducial markers.

17. A method for controlling a surgical robotic system, the method comprising:
capturing a video feed of a surgical instrument through a laparoscopic camera, wherein the surgical instrument includes a plurality of fluorescent fiducial markers;
operating an image processing device coupled to the laparoscopic camera in a white light imaging mode and a low visibility imaging mode;
processing the video feed of the surgical instrument in the low visibility imaging mode to detect the plurality of fluorescent fiducial markers;
generating an overlay of the surgical instrument;
rendering the overlay, while in the low visibility imaging mode, on a portion of the video feed including the surgical instrument, based on locations of the identified plurality of fluorescent fiducial markers; and
displaying on a screen the video feed in the low visibility imaging mode with the overlay.

18. The method according to claim 17, further comprising:
receiving user input at a surgeon console including a handle controller, wherein the surgical instrument is coupled to a robotic arm through an instrument drive unit; and
actuating the surgical instrument through the instrument drive unit in response to the user input.

19. The method according to claim 18, further comprising:
tracking a position of the surgical instrument; and
updating a location of the overlay on the video feed based on the position of the surgical instrument.

20. The method according to claim 19, wherein tracking movement of the surgical instrument is based on kinematics data of the robotic arm and the plurality of plurality of fluorescent fiducial markers.
